(19) United States
(12) Patent Application Publication
(10) Pub. No.: US 2004/0082914 A1
(43) Pub. Date: Apr. 29, 2004

(54) System and method for delivering medications

(76) Inventor: Sandra Marie Hooper, Allen, TX (US)

Correspondence Address: R. ROSS VIGUET, FULBRIGHT AND JAWORSKIL, 2200 ROSS AVENUE, SUITE 2800, DALLAS, TX, 75201-2784, US

(21) Appl. No.: 10/281,520

(22) Filed: Oct. 28, 2002

(51) Int. Cl. A61M 5/178

(52) U.S. Cl. 604/164.01; 264/209.1

(57) ABSTRACT

The invention relates to a system and method for localized drug delivery. The system includes a catheter and stylet. The catheter has a set of inwardly extending portions that function to provide structure to the catheter wall, prevent collapse or closure of the catheter lumen, and enable easy withdrawal of the stylet. The inwardly extending portions may take the form of substantially triangular shapes, semicircular, circular or other shapes. With the present invention, a catheter may effectively be installed in a patient in a proper location. The location may be tested through the transfer of fluid through the catheter while the stylet is in place during surgery. The catheter may be tightly sutured in place and the stylet easily withdrawn.

### TECHNICAL FIELD OF THE INVENTION

This invention, in general, relates to a system for drug delivery. More specifically, the invention relates to an implantable catheter system for in vivo drug delivery.

### BACKGROUND OF THE INVENTION

Many diseases and syndromes can benefit from local drug delivery. These diseases and syndromes include localized infections, cancers, chronic pain and muscle disorders, and epilepsy, among others. Antibiotics, anesthetics, morphates, and anti-seizer medications, among others, may be delivered in smaller doses to localized areas. In this manner, drugs may be delivered in effective doses without inundating the body with excess medication.

The methods may also permit delivery of drugs to regions of the body that would be inaccessible by other techniques because of membrane barriers and other physical barriers. For example, chronic lower back pain and failed back syndrome may be treated through implanted intrathecal drug administration.

Typical systems include a pump connected to a catheter. The pump may be implanted or external to the body. The distal end of the catheter may be placed near the treatment area. Typically, to hold the catheter in place, the catheter is sutured to tissues near the location of treatment. A loop is often sutured nearby to allow for movement of the patient without tearing the stationary suture.

However, several problems exist with the typical drug delivery system. These problems include difficulties in removal of stylets from the lumen of the catheter, in placing the catheter, and with blocked or collapsed catheters.

FIG. 1 represents a typical catheter system 10. The catheter includes a catheter wall 12 having an inner surface 16. During placement, a stylet 14 is often located in the lumen of the catheter. Bends and curves along the tortuous path of catheter placement often cause the stylet 14 to touch the catheter's inner surface 16. FIG. 2 depicts a large contact area 18 that may result in excess friction and difficulty in moving the stylet within the catheter including removing the stylet from the catheter.

When installing or inserting the catheter, it is often preferable to test the fluid in which the distal end of the catheter resides. This fluid may give an indication of the location of the distal end of the catheter. For example, when a catheter is placed in the spinal column, a doctor may test for spinal fluid to determine that the distal end of the catheter is located in the appropriate cavity so that the drug will be delivered to the preferred region or area. However, present catheter/stylet systems prevent or limit the drawing of fluid during the placement of the catheter.

Once the catheter is in place, the surgeon typically places a tight suture around the catheter and connects it to a tissue in the region to hold the catheter in place. Often, if the suture is too tight, the catheter will collapse, preventing both withdrawal of fluid and drug delivery through the catheter. FIG. 3A depicts the catheter 32 being sutured at a suture location 36 with a suture 34. In FIG. 3B, the suture location 36 is shown in cross-section. The suture 34 compresses the catheter radially, effectively diminishing or collapsing the lumen.

Similarly, when the loop is placed in the patient and sutured to tissue, kinks may form over time because of patient movement. In either case, the blockage of the drug delivery path may lead to pump failure and a failure of the treatment.

Once a catheter is in place, the stylet may be removed. However, because of tight sutures, loops, bends and the length of the catheter, removal of the stylet may be difficult. The tortuous path of the catheter and stylet leads to a large contact surface area between the stylet and the inner wall of the catheter. This contact area may create friction and make removal of the stylet difficult.

As such, many typical localized drug delivery systems suffer from deficiencies in catheter performance. Many other problems and disadvantages of the prior art will become apparent to one skilled in the art after comparing such prior art with the present invention as described herein.

### SUMMARY OF THE INVENTION

Aspects of the invention may be found in a system for transferring fluid to a localized area. The system may include a catheter having a set of inwardly extending portions along the inner surface of the catheter wall. These inwardly extending portions may be formed from variances in wall thickness and may take various shapes including predominantly triangular, bell-curved like, semicircular, circular, or combinations of these, among others. The system also includes a stylet located in the lumen of the catheter, the stylet contacting at least one of these inwardly extending portions. In effect, the inwardly extending portions create channels that operatively permit extraction of fluid while the stylet is located in the lumen of the catheter or the insertion of fluid. The fluid, such as radio-opaque fluid, can aid in locating the catheter during surgical procedures. The inwardly extending portions also effectively prevent the collapse of the catheter after removal of the stylet. These inwardly extending portions may be formed longitudinally along the inner surface of the wall and they may be parallel to the access of the catheter or spiral, among others. They may be placed in regular or equidistant locations about the radius of the inner surface. In addition, the channel between the successive inwardly extending portions may be small enough to prevent the stylet from entering or catching in the channel. There may be three or more inwardly extending portions. As such, there may be three or five or more inwardly extending portions.

The catheter may be formed from silicon, a radio-opaque material, and/or a bizmuth loaded material. The stylet may be formed of stainless steel or Teflon-coated stainless steel, among others.

Further aspects of the invention may be found in a method for transferring fluid to a localized area within a patient. The method may include positioning the distal end of a catheter near a treatment area. The catheter may take the form described above. The catheter may be sutured in place and the stylet removed. The method may include drawing fluid through the lumen of the catheter while the stylet is in place. The method may further include attaching an implantable drug delivery system to the end of the catheter opposite the distal ends. In one example, the catheter is placed intrathecally.

Further aspects of the invention may be found in a catheter having at least five inwardly projecting portions that extend longitudinally through the catheter along the inner surface of the wall and define a lumen. A stylet may be located within the lumen and contact the inner surface of the catheter only at the peaks of the at least five inwardly projecting portions. The inwardly projecting portions may extend parallel to the axis along the inner surface of the catheter or spiral along the inner surface of the catheter. Further, the catheter may maintain at least a portion of the lumen when compressed, bent, or sutured in place.

Additional aspects of the invention may be found in a method for manufacturing a catheter. The method includes the step of extruding a material through a die. The material may take the form of silicone. The die has a shape such that a catheter is formed with an inner wall surrounding a lumen. The inner wall is characterized by a set of inwardly extending portions. A stylet is then inserted into the lumen. The inwardly extending portions may form channels that permit fluid to be transferred while the stylet is in place. Further, the inner wall may have five or more inwardly extending portions.

As such, a system for localized drug delivery is described. Other aspects, advantages, and novel features of the present invention will become apparent from the detailed description of the invention when considered in conjunction with the accompanying drawings.

### BRIEF DESCRIPTION OF THE DRAWINGS

For a more complete understanding of the present invention and advantages thereof, reference is now made to the following description taken in conjunction with the accompanying drawings in which like reference numbers indicate like features and wherein:

FIG. 1 is a schematic diagram of prior art showing a typical catheter;

FIG. 2 is a schematic diagram of prior art showing a typical catheter;

FIGS. 3A and 3B are prior art schematics depicting a typical catheter under compression by a suture;

FIG. 4A is a schematic diagram depicting one embodiment of the present invention;

FIG. 4B is a schematic diagram depicting one embodiment of the present invention;

FIG. 4C is a schematic diagram depicting an exemplary embodiment of the invention as seen in FIG. 4A;

FIGS. 5A and 5B are schematic diagram depicting exemplary embodiments of the invention as seen in FIG. 4A;

FIG. 6 is a schematic diagram depicting an exemplary embodiment of the present invention as seen in FIG. 5B;

FIG. 7 is a schematic diagram depicting an exemplary embodiment of the present invention as seen in FIG. 5B;

FIG. 8 is a schematic diagram depicting an exemplary embodiment of the invention;

FIG. 9 is a block flow diagram depicting an exemplary method according to the invention; and

FIG. 10 is a block flow diagram depicting an exemplary method according to the invention.

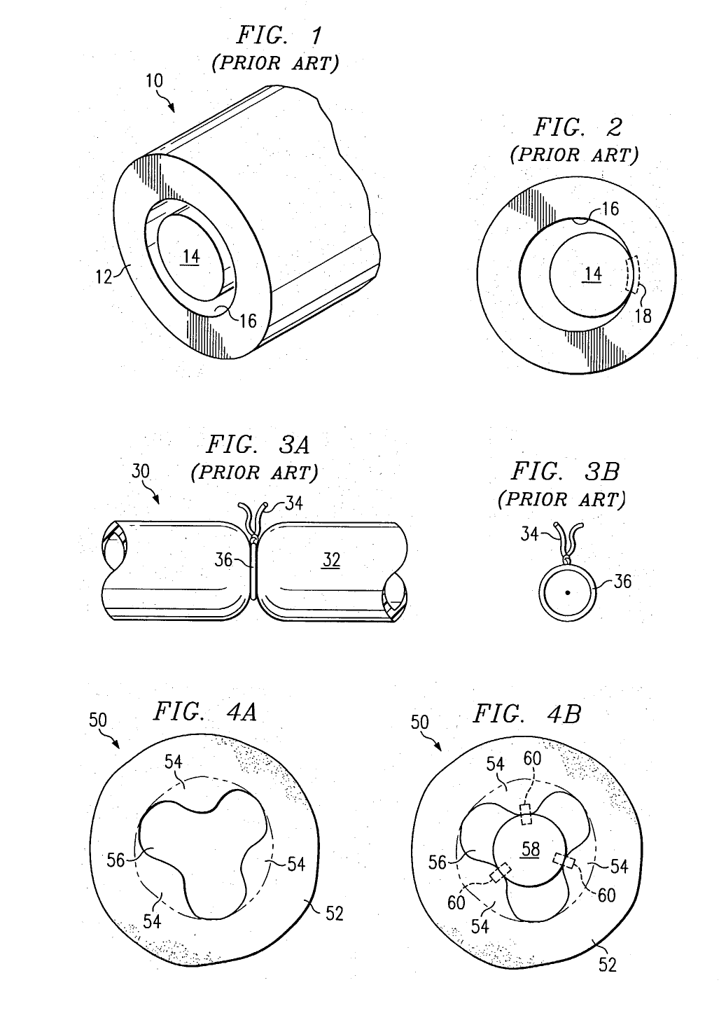

### DETAILED DESCRIPTION OF THE INVENTION

Localized drug delivery offers many advantages in the treatment of chronic pain and other diseases and syndromes. These advantages include minimizing dosages and limiting the treatment area, among others. However, many typical drug delivery system suffer from deficiencies in maintaining a clear and open channel to the treatment area and in performing the procedure for placing catheters in the appropriate locations.

The present invention includes inwardly extending portions along the inner surface of the catheter. These inwardly extending portions provide a stylet a passage through the catheter while minimizing the contact area with the interior wall of the catheter. Further, these inwardly extending portions provide channels for fluid transfer (either injected or withdrawn) during placement of the catheter such as fluid extraction or for placement of radio-opaque material to aid in locating the catheter during surgical procedures.

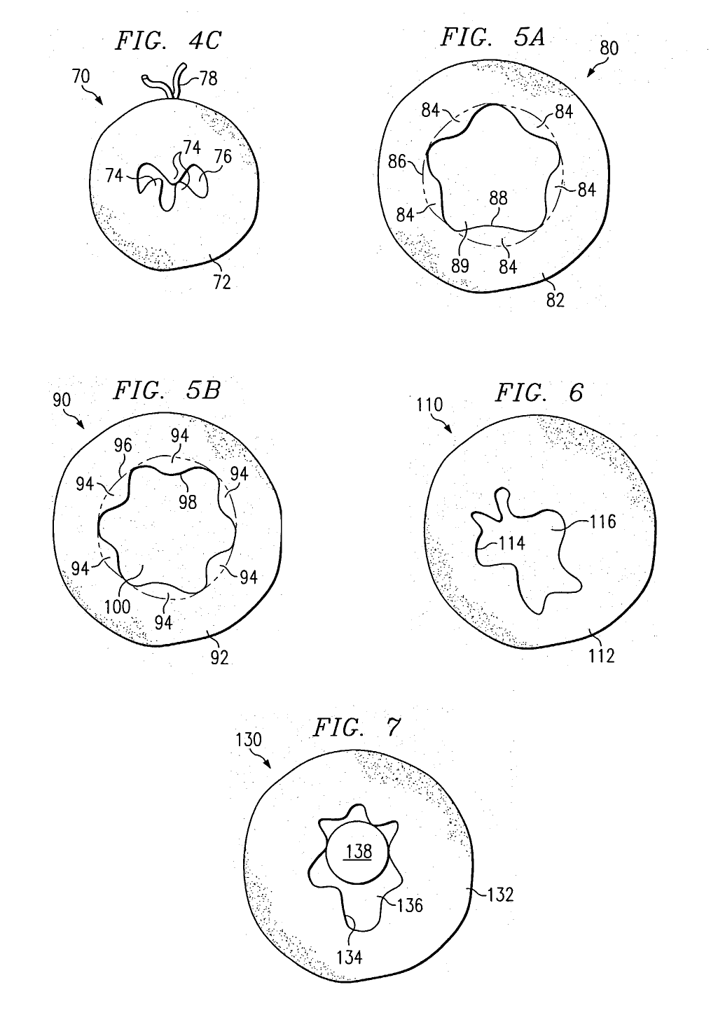

FIG. 4A depicts an exemplary embodiment of the catheter, according to the invention. In this embodiment, the catheter 50 has a catheter wall 52. Extending from the wall are inwardly extending portions 54. These portions define a lumen 56. The inwardly extending portions may also extend along the inner wall of the catheter, longitudinally or in a spiral.

The catheter wall 52 may be constructed of silicone, a radio opaque material, a bizmuth doped material, or a composite material, among others. The inwardly extending portions 54 may be formed of the same material as the wall 52. Alternately, the inwardly extending portions may be formed of another material. For example, the catheter wall 52 with inwardly extending portions 54 may be extruded in silicone. However, various materials and construction techniques, embodiments of which are described below, may be envisaged.

The catheter size may range from 3 to 34 French (0.003-0.445 inches outside diameter). For example, an interthecal or intraspinal catheter may have a size of approximately 4 French (50 to 55 thousandths of an inch in outside diameter. A cardiovascular catheter may have a size of approximately 14 to 15 French. In one exemplary embodiment, the inner most part of the inwardly extending portions may be at a radius of 14 thousandths of an inch with the inwardly extending portions extending 3 thousandths of an inch. However, various sizes may be used for various applications.

FIG. 4B depicts an exemplary embodiment of the invention with a stylet inserted therein. The stylet 58 may be inserted in lumen 56. The contact surface area of the stylet with the inwardly extending portions is limited to a small area 60. In addition, channels 56 between the inwardly extending portions 54 are formed, permitting transfer of fluid while the stylet is in place.

The stylet may be formed from various materials including surgical steel, stainless steel, Teflon coated stainless steel, various plastics and composite materials, among others. However, the stylet may be formed using various materials and manufacturing techniques.

FIG. 4C shows the exemplary embodiment of FIG. 4A when compressed radially by a suture 78 or another compressive force. The catheter wall 72 is compressed, effectively diminishing the lumen. However, interference with the inwardly extending portions 74 effectively maintains a diminished channel or channels along the catheter.

In this manner, the inwardly extending portions function to interfere with one another, preventing collapse. The inwardly extending portions also provide structural support about the lumen. In addition, spacing between the inwardly extending portions provides channels for transferring fluid while the stylet is in place.

FIG. 5A depicts another exemplary embodiment of the catheter according to the invention. The catheter wall 82 has an inner surface defined by at least five inwardly extending portions 84. These inwardly extending portions define the cross-sectional shape of the lumen 89.

FIG. 5B depicts another exemplary embodiment of the catheter according to the invention. The wall of the catheter includes at least six inwardly extending portions 94 defining an inner surface of the wall 98, which defines lumen 100. Having either more inwardly extending portions or larger inwardly extending portions, the channel between portions is diminished in size such that the stylet is prevented from fitting within a channel. In this manner, the size, number and arrangement of the inwardly extending portions may be selected to prevent a stylet from moving between inwardly extending portions.

FIG. 6 depicts an exemplary embodiment of the catheter as seen in FIG. 5B under compression. Compression of the wall 112 leads to a distorted inner surface 114 and a diminished lumen 116. However, interference between inwardly extending portions and the structural integrity that they provide to the catheter prevents the lumen from being wholly closed, thus, maintaining a channel for fluid transfer. FIG. 7 depicts a similar compression or distortion with the presence of a stylet. Here, to, the wall 132 of the catheter 130 is compressed, distorting the inner surface of the catheter 134. The inner surface of the catheter then touches the stylet 138 at various peak locations of a few of the inwardly extending portions. As such, the lumen 136 is maintained and the stylet contacts the inner surface 134 of the catheter wall 132 at limited contact points. In effect, the fluid may be transferred through the catheter while the stylet is in place and the stylet may be removed with minimal friction.

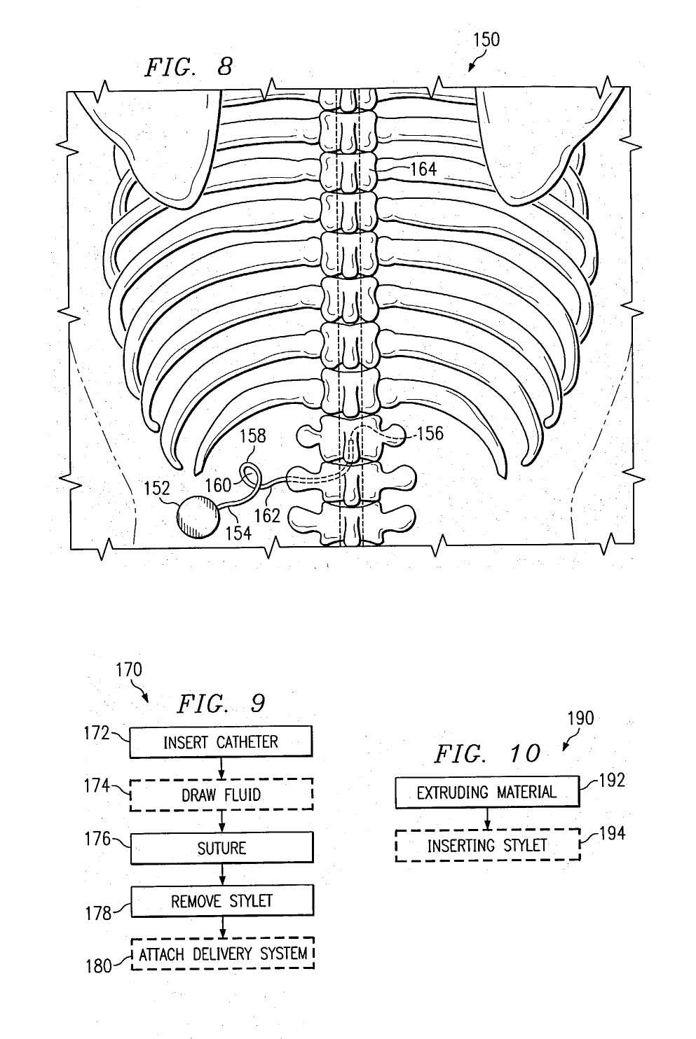

FIG. 8 depicts an exemplary placement of the catheter in a patient. The catheter 154 is placed such that the distal end 156 is located about a treatment area. In this case, the treatment area is near the spinal cord within the spinal column 164. The catheter 154 is sutured at location 162 and a loop 158 of the catheter may be sutured nearby at a point 160. The catheter 154 is attached to an implantable drug pump 152. Alternately, a drug pump 152 may be located external to the body. In this manner, treatment may be delivered through the catheter 154 to the treatment area 156 without blockages caused by either the sutures 162, bends, loops, or kinks in the catheter.

FIG. 9 is a block flow diagram depicting an exemplary method of the present invention for using one embodiment of the present invention. The method 170 begins with the insertion of the catheter as seen in block 172. To determine the location of the distal end of the catheter, a surgeon may draw fluid through the catheter while the stylet is in place as seen in block 174. For example, a surgeon may test for spinal fluid when inserting a catheter close to the spinal cord.

Once the catheter is in place, the surgeon sutures the catheter to tissue near the treatment location as seen in block 176. For example, the surgeon may suture the catheter to a location outside of the spine near the location where the catheter is inserted into the spine.

With the catheter in place, the stylet is removed as seen in block 173. The inwardly extending portions act to limit contact surface area and enable the stylet to be removed with minimal force.

Once the stylet is removed, a drug delivery system such as drug pump may be attached as seen in block 180. The system may be implanted into the patient or extend outside the patient.

However, the various method steps may be performed in varying sequences. For example, the stylet may be withdrawn prior to the suturing. In another exemplary embodiment, the surgeon may not withdraw fluid.

FIG. 10 depicts a method for manufacturing a catheter. The method includes the step of extruding a material through a die as seen in block 192. The material may take the form of silicone. The die has a shape such that a catheter is formed with an inner wall surrounding a lumen. The inner wall is characterized by a set of inwardly extending portions. A stylet is then inserted into the lumen as seen in a block 194. The inwardly extending portions may form channels that permit fluid to be transferred while the stylet is in place. Further, the inner wall may have five or more inwardly extending portions.

As such, a system and method for localized drug delivery is described. In view of the above detailed description of the present invention and associated drawings, other modifications and variations will now become apparent to those skilled in the art. It should also be apparent that such other modifications and variations may be effected without departing from the spirit and scope of the present invention as set forth in the claims, which follow.

What is claimed is:

1. A system for transferring fluid to a location internal to a patient, the system comprising:
a catheter having a wall, the wall having an inner surface defining a lumen, the inner surface having a plurality of inwardly-extending portions; and
a removable stylet located in the lumen.

2. The system of claim 1, wherein the plurality of inwardly-extending portions operatively permit extraction of fluid while the removable stylet is located in the lumen of the catheter.

3. The system of claim 1, wherein the plurality of inwardly-extending portions operatively permit injection of radio-opaque fluid into the catheter while the removable stylet is located in the lumen of the catheter.

4. The system of claim 1, wherein the plurality of inwardly-extending portions operatively prevents collapse of the catheter after removal of the removable stylet from the lumen of the catheter.

5. The system of claim 1, wherein the plurality of inwardly-extending portions are formed longitudinally along the inner surface of the wall of the catheter.

6. The system of claim 1, wherein the plurality of inwardly-extending portions are formed as a longitudinally extending spiral along the inner surface of the wall of the catheter.

7. The system of claim 1, wherein the plurality of inwardly-extending portions are distributed about the radius at equidistant locations.

8. The system of claim 1, wherein the plurality of inwardly-extending portions are spaced about the radius such that a distance from the peak of one inwardly extending portion to the peak of an adjacent inwardly extending portion is less than the radius of the removable stylet.

9. The system of claim 1, wherein the plurality of inwardly-extending portions comprise at least five inwardly extending portions.

10. The system of claim 1, wherein the catheter is formed from material comprising silicone.

11. The system of claim 1, wherein the catheter is formed from radio-opaque material.

12. The system of claim 1, wherein the catheter is formed from bizmuth-loaded material.

13. The system of claim 1, wherein the removable stylet is formed of stainless steel.

14. The system of claim 1, wherein the removable stylet is formed of Teflon coated stainless steel.

15. A method for transferring fluid to a location internal to a patient, the method comprising:
positioning a distal end of a catheter about a treatment area, the catheter having a wall, the wall having an inner surface defining a lumen, the inner surface having a plurality of inwardly-extending portions; a removable stylet located within the lumen;
suturing the catheter to a tissue; and
removing the removable stylet.

16. The method of claim 15, further comprising:
drawing fluid through the lumen of the catheter while the removable stylet is located in the lumen.

17. The method of claim 15, further comprising:
attaching an implantable drug delivery system to an end of the catheter opposite the distal end.

18. The method of claim 15, wherein the treatment area is intrathecal.

19. A catheter for drug delivery, the catheter comprising:
a wall having an inner surface defining a lumen, the inner surface having at least five inwardly-projecting portions that extend longitudinally through the catheter; and
a removable stylet located within the lumen of the catheter, a contact area associated with the removable stylet being limited by peaks of the at least five inwardly projecting portions.

20. The catheter of claim 19, wherein the at least five inwardly-projecting portions form a longitudinal spiral along the inner surface of the catheter.

21. The catheter of claim 19, wherein upon radial compression, the inwardly-projecting portions interfere to maintain at least a portion of the lumen.

22. A method for manufacturing a catheter, the method comprising:
extruding material through a die, the die having a shape such that a catheter is formed having an inner wall surrounding a lumen, the inner wall comprising inwardly extending portions; and
inserting a stylet into the lumen of the catheter.

23. The method of claim 22, wherein the inner wall comprises at least five inwardly extending portions.

24. The method of claim 22, wherein the inwardly extending portions form channels through which fluid may be transferred when the stylet is located in the lumen of the catheter.
